(19) United States
(12) Patent Application Publication
(10) Pub. No.: US 2008/0292599 A1
(43) Pub. Date: Nov. 27, 2008

(54) Chondrogenic Compositions and Methods of Use

(75) Inventors: Susan J. Drapeau, Cordova, TN (US); Sangwook T. Yoon, Decatur, GA (US)

Correspondence Address: MEDTRONIC;Attn: Noreen Johnson - IP Legal Department, 2600 Sofamor Danek Drive, MEMPHIS, TN, 38132, US

(73) Assignee: Warsaw Orthopedic, Inc., Warsaw, IN (US)

(21) Appl. No.: 12/127,586

(22) Filed: May 27, 2008

Related U.S. Application Data
(63) Continuation-in-part of application No. 11/443,387, filed on May 30, 2006, now abandoned.
Application No. 11/443,387 is a continuation-in-part of application No. 10/382,844, filed on Mar. 7, 2003.
Application No. 10/382,844 is a continuation-in-part of application No. 10/292,951, filed on Nov. 13, 2002.
Application No. 10/292,951 is a continuation-in-part of application No. 10/806,915, filed on Mar. 23, 2004.
Application No. 10/806,915 is a continuation-in-part of application No. 09/959,578, filed on Jun. 21, 2002, now Pat. No. 7,045,614.
(60) Provisional application No. 60/685,224, filed on May 27, 2005.
(60) Provisional application No. 60/331,321, filed on Nov. 14, 2001.
(60) Provisional application No. 60/132,021, filed on Apr. 30, 1999.

(51) Int. Cl. A61K 35/12; C12N 5/08; A61P 19/00

(52) U.S. Cl. 424/93.7; 435/366

(57) ABSTRACT

The invention provides a method of treatment joint and disk disease comprising administering to the subject in need thereof a novel composition comprising a substantially purified plurality of cells enhanced with at least one bioactive factor capable of causing at least a portion of the plurality of cells to express an increased amount of at least one chondrogenic marker.

### CROSS-REFERENCE TO RELATED APPLICATIONS

This application is a continuation of U.S. application Ser. No. 11/443,387 filed on May 30, 2006 which claims the benefit of provisional application 60/685,224, filed on May 27, 2005, which is incorporated herein by reference. This application is also a continuation-in-part of a U.S. application Ser. No. 10/382,844 filed on Mar. 7, 2003, which is a continuation-in-part of U.S. patent application Ser. No. 10/292,951, filed Nov. 13, 2002, pending, which claims priority to U.S. Provisional Application Ser. No. 60/331,321, filed Nov. 14, 2001. This application is also a continuation-in-part of a U.S. application Ser. No. 10/806,915 filed on Mar. 23, 2004, which is a continuation-in-part of U.S. application Ser. No. 09/959,578 filed on Jun. 21, 2002, now U.S. Pat. No. 7,045,614, and which is a national stage entry of Application PCT/US00/1164 filed on Apr. 28, 2000 and which claims the benefit of U.S. Provisional Application 60/132,021 filed on Apr. 30, 1999.

### FIELD OF THE INVENTION

The invention relates to compositions and methods of providing relatively easily-obtained chondrocyte-like cells to a disc to upregulate matrix production so as to prevent further degeneration of the disc.

### BACKGROUND

The progressive degeneration of intervertebral discs with age is believed to be associated with a decrease in cell density and a decrease in synthesis of cartilage-specific matrix components, especially proteoglycans. See, e.g., S. J. Lipson & H. Muir H., Volvo award in basic science: Proteoglycans in Experimental Intervertebral Disc Degeneration, 6 SPINE 194-210 (1981); A. G. Nerlich et al., Volvo Award winner in basic science studies: Immunohistologic Markers for Age-related Changes of Human Lumbar Intervertebral Discs, 22 SPINE 2781-95 (1997); R. H. Pearce et al., Degeneration and the Chemical Composition of the Human Lumbar Intervertebral Disc, 5 J. ORTHOP. RES. 198-205 (1987). One approach to address these decreases in proteoglycans is to implant autologous disc cells into a degenerated disc. It is thought that these implanted cells would stimulate the disc by upregulating matrix production. Implantation of autologous disc cells has been tested and shown to be technically feasible and biologically relevant to repairing disc damage and retarding disc degeneration. See T. Ganey et al., Disc Chondrocyte Transplantation in a Canine Model: A Treatment for Degenerated or Damaged Intervertebral Disc, 28 SPINE 2609-20 (2003). However, the donor site morbidity of autologous cell harvest and the risk of immunological effects limits the use of these approaches in clinical practice.

### SUMMARY OF THE INVENTION

It is therefore an object of the invention to provide relatively easily-obtained chondrocyte-like cells to a disc to upregulate matrix production so as to prevent further degeneration of the disc.

In one aspect, the invention provides a composition comprising a substantially purified plurality of cells enhanced with at least one bioactive factor capable of causing at least a portion of the plurality of cells to express an altered amount of at least one chondrogenic marker. In different embodiments, of the invention, the members of the plurality of cells are selected from the group consisting of bone marrow cells, adipose cells, and muscle cells. Further, in one embodiment of the invention, the at least one bioactive factor is LMP-1.

In another aspect, the invention provides a formulation comprising the composition described above in combination with a suitable carrier or diluent. In different embodiments of the invention, the composition of the present invention is in a liquid or semi-solid carrier suitable for intramuscular, intravenous, intramedullary, or intraarticular injection.

In another aspect, the invention provides a method of treatment of a chondrocyte-derived tissue comprising administering to a subject in need thereof an effective amount of the composition according to any of the embodiments described above. In different embodiments of the invention, the members of the plurality of cells are cultured under conditions promoting their differentiation into chondrogenic cells.

In yet another aspect, the invention provides a method of treatment of a chondrocyte-derived tissue comprising administering to a subject in need thereof an effective amount of a vector encoding the at least one bioactive factor. In one embodiment, the at least one bioactive factor is LMP-1.

### BRIEF DESCRIPTION OF THE DRAWINGS

FIG. 1 is a bar graph demonstrating the effect of Ad-f35-LMP1 on sulfated-glycosaminoglycans (sGAG) production by disc cells. Data are normalized to sGAG values for the control group (Mean ±SEM, n=6 each). The symbol “*” indicates P<0.05 versus the control group.

FIG. 2 is a bar graph demonstrating the effect of Ad-LMP1-GFP on LMP-1 mRNA levels by disc cells. Data are normalized to the mRNA expression for the control group (Mean ±SD, n=6 each). The symbol “*” indicates P<0.05 versus the control group.

FIG. 3 is a bar graph demonstrating the effect of Ad-f35-LMP1 on aggrecan mRNA level by disc cells. Data are normalized to aggrecan values for the control group (Mean ±SEM, n=6 each). The symbol “*” indicates P<0.05 versus the control group.

FIG. 4 is a bar graph demonstrating the effect of Ad-f35-LMP1 on BMP-2 mRNA levels by disc cells. Data are normalized to BMP-2 mRNA expression for the control group (Mean ±SEM, n=6 each). The symbol “*” indicates P<0.05 versus the control group.

FIG. 5 is a bar graph demonstrating the effect of Ad-f35-LMP1 on BMP-7 mRNA levels by disc cells. Data are normalized to BMP-7 mRNA expression for the control group (Mean ±SEM, n=6 each). The symbol “*” indicates P<0.05 versus the control group.

FIG. 6 is a bar graph demonstrating the effect of Ad-f35-LMP1 on BMP-9 mRNA levels by disc cells. Data are normalized to BMP-9 mRNA expression for the control group (Mean ±SEM, n=6 each). The symbol “*” indicates P<0.05 versus the control group.

### DETAILED DESCRIPTION

For the purposes of this invention, the following non-limiting definitions are provided:

As used herein, the term “cartilage” refers to joint spaces, intervertebral discs, and all cartilaginous tissues within the human body.

The terms “allograft” and “allogeneic” refer to a graft of tissue obtained from a donor of the same species as, but with a different genetic make-up from, the recipient, as a tissue transplant between two humans.

The terms “autograft” and “autogeneic” refer to being derived or transferred from the same individual's body.

The terms “xenograft” and “xenogeneic” refer to being derived from a donor of a different species than recipient.

The terms “intervertebral disc” and “intervertebral disc tissue” include the endplate, the nucleus pulposis and/or the annulus fibrosis.

The term “vector” refers to a nucleic acid assembly capable of transferring gene sequences to target cells (e.g., viral vectors, non-viral vectors, particulate carriers, and liposomes). The term “expression vector” refers to a nucleic acid assembly containing a promoter which is capable of directing the expression of a sequence or gene of interest in a cell. Vectors typically contain nucleic acid sequences encoding selectable markers for selection of cells that have been transfected by the vector. Generally, “vector construct,” “expression vector,” and “gene transfer vector,” refer to any nucleic acid construct capable of directing the expression of a nucleic acid sequence of interest and which can transfer gene sequences to target cells. Thus, the term includes cloning and expression vehicles, as well as viral vectors.

The term “treating” or “treatment” of a disease refers to executing a protocol, which may include administering one or more drugs to a patient (human or otherwise), in an effort to alleviate signs or symptoms of the disease. Alleviation can occur prior to signs or symptoms of the disease appearing, as well as after their appearance. Thus, “treating” or “treatment” includes “preventing” or “prevention” of disease. In addition, “treating” or “treatment” does not require complete alleviation of signs or symptoms, does not require a cure, and specifically includes protocols which have only a marginal effect on the patient.

The term “practitioner” refers to a person who uses methods and compositions of the current invention on the patient. The term includes, without limitations, doctors, nurses, scientists, and other medical or scientific personnel.

The term “multipotent cells” refers to cells capable of differentiation into more than one cell type. As used herein, multipotential cells include but are not limited to mesenchymal cells.

As used herein, the term “LMP-1” includes bioactive fragments, derivatives and analogs thereof, capable of causing the members of the plurality of cells to express at least one chondrogenic marker. LMP-1 also includes LMP as used in the U.S. Pat. 20030125248 (Hair) and LMP splice variants including but not limited to those disclosed in WO00/66178 (from application PCT/US00/11664). As used herein LMP also includes LIM Mineralization Protein as disclosed in the U.S. Pat. 20030180266 (McKay). The entire teachings of all of the above publications are incorporated herein by reference.

As has been discussed in U.S. Pat. 20030180266, over-expression of HLMP-3 in secondary rat osteoblast cultures induced bone nodule formation (287±56) similar to the effect seen for glucocorticoid (272±7) and HLMP-1 (232±200). Since HLMP-3 lacks the C-terminal LIM domains, there regions are not required for osteoinductive activity.

Over-expression of HLMP-2, however, did not induce nodule formation (11±3). These data suggest that the amino acids encoded by the deleted 119 base pairs (encoding amino acids 94-133 of LMP-1) are necessary for osteoinduction.

Additionally, a truncated (short) version of HLMP-1, termed HLMP-1s, has been characterized. This short version resulted from a point mutation in one source of a cDNA clone, providing a stop codon which truncates the protein. HLMP-1s has been found to be fully functional when expressed in cell culture and in vivo.

The methods of the present invention utilize routine techniques in the field of molecular biology. Basic texts disclosing general molecular biology methods include Sambrook et al., Molecular Cloning, A Laboratory Manual (3d ed. 2001) and Ausubel et al., Current Protocols in Molecular Biology (1994).

In one aspect, the invention provides a composition comprising a substantially purified plurality of cells enhanced with at least one bioactive factor capable of causing at least a portion of the plurality of cells to differentiate into chondrogenic or chondrogenic-like cells.

The plurality of cells of the present invention may be stumulated to produce the at least one chondrogenic marker by the at least one bioactive factor. The at least one bioactive factor comprises a molecule capable of causing the members of the plurality of cells to express at least one chondrogenic marker, either directly or indirectly. For example, it is known that bone morphogenic proteins 2, 7, and 9 (BMP-2, BMP-7 and BMP-9, respectively) induce expression of proteoglycans. Thus, if a molecule induces expression of these and other BMPs which, in turn, induce expression of proteoglycans, such molecule is considered the at least one bioactive factor. The at least one bioactive factor may, in some embodiments, refer to amino-acid sequences, bioactive fragments, derivatives and analogs thereof. In other embodiments, the at least one bioactive factor comprises a nucleic acid sequence comprising a nucleic acid sequence encoding the molecule capable of causing the members of the plurality of cells to express the at least one chondrogenic marker, either directly or indirectly. Thus, a vector, e.g., a retroviral vector, comprising a nucleic acid encoding LMP-1 or a fragment thereof capable of causing the members of the plurality of cells to express at least one chondrogenic marker, either directly or indirectly, is within the meaning of the term “at least one bioactive factor.”

In the literature, one approach to stimulate proteoglycans synthesis is to use cytokines. Preliminary work with several candidate cytokines such as transforming growth factor-beta (TGF-β1), BMP-2 and BMP-7 has shown that they can stimulate aggrecan synthesis rates in disc cells. See S. T. Yoon, The Potential of Gene Therapy for the Treatment of Disc Degeneration, 35 ORTHOP. CLIN. N. AM. 95-100 (2004); Y. Zhang et al., Growth Factor Osteogenic Protein-1: Differing Effects on Cells from Three Distinct Zones in the Bovine Intervertebral Disc, 83 AM. J. PHYS. MED. REHABIL. 515-21 (2004); J. Yung Lee et al., New Use of a Three-dimensional Pellet Culture System for Human Intervertebral Disc Cells: Initial Characterization and Potential Use for Tissue Engineering, 26 SPINE 2316-22 (2001).

A person of ordinary skill in the art will recognize that proteoglycans, including aggrecan, are not the only suitable chondrogenic markers. In different embodiments of the invention, the at least one chondrogenic marker is selected from the group consisting of Collagen Type II, proteoglycans such as aggrecan, versican, or fibromodulin, lumican, SOX-9, sulfated-glycosaminoglycans, chondrocyte proliferation, cell condensation, alkaline phosphatase, Collagen Type X, and any combination thereof., and any combination thereof.

In one embodiment of the invention, the at least one bioactive factor comprises LIM mineralization protein-1 (LMP-1). LMP-1 is a novel, highly conserved intracellular regulator protein, which has been shown by the Applicants to increase proteoglycan production by upregulating multiple BMPs. See S. T. Yoon et al., ISSLS Prize Winner: LMP-1 Upregulates Intervertebral Disc Cell Production of Proteoglycans and BMPs In Vitro and In Vivo, 29 SPINE 2603-11 (2004). It is believed that LMP-1 may be a good candidate for the treatment of degenerated discs by upregulating the synthesis of proteoglycans or other relevant extracellular matrix molecules.

A person of ordinary skill in the art will undoubtedly recognize that cells suitable for the present invention, as described below, may be transformed by a nucleic acid sequence comprising a nucleic acid sequence encoding at least one bioactive factor. The nucleic acid sequence comprising the nucleic acid sequence encoding the at least one bioactive factor may be introduced into the cells by multiple ways. Suitable methods of introducing exogenous nucleic acid sequences are described in Sambrook and Russel, Molecular Cloning: A Laboratory Manual (3^{rd} Edition), Cold Spring Harbor Press, NY, 2000. These methods include, without limitation, physical transfer techniques, such as, for example, microinjection or electroporation; transfections, such as, for example, calcium phosphate transfections; membrane fusion transfer, using, for example, liposomes; and viral transfer, such as, for example, the transfer using DNA or retroviral vectors. Other methods for introducing the nucleic acid sequences of the present invention into suitable cells, such as, for example, electroporation (see, e.g., Iversen et al., Electroporation by nucleofector is the best nonviral transfection technique in human endothelial and smooth muscle cells, GENETIC VACCINES AND THER. 3:2-14 (2005)) will be apparent to a person of ordinary skill in the art. All such methods are within the scope of the present invention.

In one embodiment, the nucleic acid encoding for the at least one bioactive factor is a viral vector. The vectors suitable for the present invention include, without limitations, plasmid vectors and viral vectors. Viral expression vectors are useful, particularly those that efficiently transduce heart cells (e.g., alphaviral, lentiviral, retroviral, adenoviral, adeno-associated viral (AAV)), as described, for example, in Williams and Koch, Annu. Rev. Physiol. 66:49 (2004); del Monte and Hajjar, J. Physiol. 546.1:49 (2003).

In one embodiment, the vector comprises an adeno-associated virus (AAV), from the parvovirus family. These vectors can insert genetic material at a specific site on chromosome 19. A person of ordinary skill in the art will recognize that among the advantages of AAV are the facts that AAV is not pathogenic and that most people treated with AAV will not build an immune response to remove the virus.

Both adenoviral and AAV vectors have been shown to be effective at delivering transgenes (including transgenes directed to desired) into heart cells, including failing cardiomycoytes (See, e.g., Iwanaga et al., J. Clin. Invest. 113:727 (2004); Seth et al., Proc. Natl. Acad. Sci. USA 101:16683 (2004); Champion et al., Circulation 108:2790 (2003); Li et al., Gene Ther. 10:1807 (2003); Vassalli et al., Int. J. Cardiol. 90:229 (2003); del Monte et al., Circulation 105:904 (2002); Hoshijima et al., Nat. Med. 8:864 (2002); Eizema et al., Circulation 101:2193 (2000); Miyamoto et al., Proc. Natl. Acad. Sci. USA 97:793 (2000); He et al., Circulation 100:974 (1999). Recent reports have demonstrated the use of AAV vectors for sustained gene expression in mouse and hamster myocardium and arteries for over one year (Li et al., Gene Ther. 10:1807 (2003); Vassalli et al., Int. J. Cardiol. 90:229 (2003)). In particular, expression vectors based on AAV serotype 6 have been shown to efficiently transduce both skeletal and cardiac muscle (e.g., Blankinship et al., Mol. Ther. 10:671 (2004)).

The nucleic acid sequence comprising the nucleic acid sequence encoding the at least one bioactive factor may be constructed by methods generally known to persons of ordinary skill in the art and described, for example, in Sambrook et al., Molecular Cloning, A Laboratory Manual (3d ed. 2001) and Ausubel et al., Current Protocols in Molecular Biology (1994). Further, the nucleic acid sequence comprising the nucleic acid sequence encoding the at least one bioactive factor of the present invention, especially in an embodiment comprising a viral vector, may be produced by multiple methods, most notably, by using packaging cell strains such as, for example, those described in J. M. Coffin, S. H. Hughes & H. E. Varmus (eds.), Retroviruses, Cold Spring Harbor Laboratory Press. Other methods for producing retroviruses and for infecting cells in vitro or in vivo are described in Current Protocols in Molecular Biology, Ausubel, F. M. et al. (eds.) Greene Publishing Associates. (1989), Sections 9.10-9.14. A person of ordinary skill in the art will undoubtedly recognize that at least in some embodiments of the invention, it will be advantageous to formulate the nucleic acid sequence in a way that increases the efficiency of transformation. By way of example only and without any limitations, the nucleic acid sequence may be placed within liposomes. The liposomes may be prepared by methods known in the art, such as described, for example, in Epstein et al., Proc. Natl. Acad. Sci. USA, 82: 3688 (1985); Hwang et al., Proc. Natl. Acad. Sci. USA, 77: 4030 (1980); the U.S. Pat. No. 4,485,045 and the U.S. Pat. No. 4,544,545. Liposomes with enhanced circulation time are disclosed in the U.S. Pat. No. 5,013,556.

Research into LMP-1 to stimulate protoglycan and BMP upregulation has previously only focused on intervertebral disc cells or bone marrow cells. However, this invention is not limited to these cell types only. Multiple cell types are useful for this invention, such as for example, different types of multipotential cells. The multipotential cells can be derived from various tissue sources in the body. In different embodiments the cell population may be isolated from a living donor or a cadaver tissue source. Such tissue sources include, but are not limited to, adipose tissue, muscle tissue, peripheral blood, cord blood, blood vessels, skeletal muscle, skin liver, and heart. In the practice of the invention, the cell source may include whole cells, concentrated cells, filtered cells, separated cells, and cell populations isolated and culture-expanded from a tissue source.

In one embodiment, the members of the plurality of cells are bone marrow cells. These cells are readily available from an accessible source and can be harvested from human donors with minimal morbidity. If the bone marrow cells are used in the practice of the invention, the cell source may be whole bone marrow, concentrated bone marrow, filtered bone marrow, separated bone marrow cells, and cell populations isolated and culture-expanded from the bone marrow source. Notably, bone marrow contains a population of mesenchymal cells. It has been reported that transplanted human mesenchymal stem cells into cartilage might undergo site-specific differentiation into chondrocytes. See K. W. Liechty et al., Human Mesenchymal Stem Cells Engraft and Demonstrate Site-specific Differentiation After In Utero Transplantation in Sheep, 6 NAT. MED. 1282-6 (2000). Importantly, using human bone marrow cells obviates the practical problems of autologous or allogeneic disc cell harvest and greatly shortens the time required for cell preparation in the clinical transplantation procedure.

Adult bone marrow cells have been shown to differentiate into chondrocytes in vitro and in vivo. See D. J. Prockop. Marrow Stromal Cells as Stem Cells for Nonhematopoietic Tissues, 276 SCIENCE 71-4 (1997); M. F. Pittenger et al., Multilineage Potential of Adult Human Mesenchymal Stem Cells, 284 SCIENCE 143-7 (1999); P. Bianco et al., Bone Marrow Stromal Stem Cells Nature, Biology, and Potential Applications, 19 STEM CELLS 180-92 (2001). Engineering of adult marrow cells to express chondrogenic genes has been reported to direct their differentiation towards cartilage in situ and hence to repair the cartilage. See N. Adachi et al., Muscle Derived, Cell Based Ex Vivo Gene Therapy for Treatment of Full Thickness Articular Cartilage Defects, 29 J. RHEUMATOL. 1920-30 (2002); Y. Gafni et al., Stem Cells as Vehicles for Orthopedic Gene Therapy, 11 GENE THER. 417-26 (2004). BMPs may promote osteogenic differentiation of mesenchymal stem cells, but due to the avascular and low oxygen tension environment within the disc, the mesenchymal stem cells are more likely to differentiate into chondrocytes. See D. A. Puleo, Dependence of Mesenchymal Cell Responses on Duration of Exposure to Bone Morphogenetic Protein-2 In Vitro, 173 J. CELL. PHYSIOL. 93-101 (1997); 0. Fromigue et al., Bone Morphogenetic Protein-2 and Transforming Growth Factor-beta2 Interact to Modulate Human Bone Marrow Stromal Cell Proliferation and Differentiation, 68 J. CELL. BIOCHEM. 411-26 (1998); A. H. Reddi, Bone Morphogenetic Proteins, Bone Marrow Stromal Cells, and Mesenchymal Stem Cells. Maureen Owen Revisited, 1995 CLIN. ORTHOP. 115-9; M. K. Majumdar et al., BMP-2 and BMP-9 Promotes Chondrogenic Differentiation of Human Multipotential Mesenchymal Cells and Overcomes the Inhibitory Effect of IL-1, 189 J. CELL. PHYSIOL. 275-84 (2001).

In summary, human bone marrow cells have the potential to be induced by LMP-1 to increase synthesis of proteoglycans and other chondrogenic markers by upregulating multiple BMPs. Thus, these cells are good candidates for ex vivo gene therapy for disc degeneration.

Accordingly, if bone marrow, in particular human bone marrow, could be stimulated to selectively differentiate into chondrocytes, either when transplanted into a joint or disc or when maintained ex vivo, then it could be used as an effective therapeutic agent.

The members of the plurality of cells of the present invention may be derived not only from an autogeneic source, but also from allogeneic or even xenogeneic sources. A person of ordinary skill in the art will understand, however, that using autogeneic source of the members of the plurality of cells will minimize chance of immune response and other unwelcome side effects to the composition of the present invention.

When transplanted into an intervertebral disc, the LMP-1 enhanced bone marrow cells may arrest and/or reverse disc degeneration. Applicants decided that by enhancing bone marrow with LMP-1, a therapeutic chondrogenic matrix would form. In one particular embodiment, if transplanted into a damaged disc, then it may be capable of repairing the disc.

In one embodiment of the invention, the bone marrow cells and mesenchymal cells transformed with LMP-1 as described above, when placed in a damaged disc, may express genes directing the differentiation of the mesenchymal stem cells towards cartilage in situ and hence to repair the cartilage of a failing disc. Still further, an upregulation of proteoglycan, in particular aggrecan, will protect the disc from further proteolytic degradation.

A person of ordinary skill in the art will undoubtedly appreciate that several methods exist for harvesting the cells to make the plurality of cells according to the instant invention. For example, the bone marrow cells, including the mesenchymal cell enriched fraction, may be harvested as described below in the “Examples” section. In another embodiment, the cells may be derived from adipose tissue. If this embodiment is selected, the cells may be purified and cultured under conditions described in the U.S. Pat. 20050282275 (Katz). Briefly, for chondrogenic differentiation, preferably the cells are cultured in high density (e.g., at about several million cells/ml or using micromass culture techniques), and also in the presence of low amounts of serum (e.g., from about 1% to about 5%). Further, the cells can be cultured on a scaffold to grow into the desired shape. A suitable non-limiting example of such method useful for treatment of meniscal repair is described, for example, in the U.S. Pat. 20050234549 (Kladakis). Further, the cells can be cultured in a bioreactor, such as, for example, the bioreactor disclosed in the U.S. Pat. No. 6,875,605.

As discussed above, the culture conditions are important for the differentiation of the members of the plurality of cells of the present invention into the chondrogenic cells. Plating with high density, e.g., between about 10 and about 10 cells/mL and culturing under low oxygen tension, e.g., between about 1% and about 5% O₂ plays a role in stimulating the cultured members of the plurality of cells to enter chondrogenic lineage. Further, a person of ordinary skill would find it advantageous to culture the members of the plurality of cells under hydrostatic pressure prior to introduction to the subject, wherein the hydrostatic pressure mimics the physical stimulation of normal activities of daily living. In one embodiment, the hydrostatic pressure ranges from about 1 to about 10 MPa.

Further, a person of ordinary skill in the art will understand that even not fully differentiated members of the plurality of cells may be administered to the subject in need thereof. Certain physical and chemical characteristics of the placement area within the subject's body will cause the members of the plurality of cells which are not fully differentiated to differentiate into chondrocytes or chondrocyte-like cells and thereby repair or form cartilage in the subject. Among those physical and chemical characteristics are compressive forces, shear forces, low oxygen tension (between about 1% and about 5%), relatively high pressure. A suitable non-limiting example of a combination of such conditions includes 1,800 cycles/day or 7,200 cycles/day of 1 Hz sinusoidal hydrostatic compression to 5 MPa. Elder et al., Cyclic hydrostatic compression stimulates chondroinduction of C3H/10T1/2 cells. BIOMECH MODEL MECHANOBIOL. 3(3):141-6 (2005). Epub Jan. 25, 2005. Joints and intervertebral disks have such characteristics, and therefore will stimulate the members of the plurality of cells to differentiate into chondrocytes or chondrocyte-like cells if the members of the plurality of cells are not fully differentiated at the time of the administration to the subject.

A person of ordinary skill in the art will appreciate that the composition of the present invention may further comprise at least one additive. Suitable examples of the at least one additive include, without limitations, lubricants, anti-inflammatory agents, antibiotics, analgesics and any combinations thereof.

Suitable examples of lubricants include, without limitations, hyaluronic acid, hyaluronan, lubricin, polyethylene glycol, and any combinations thereof.

Suitable anti-inflammatory compounds include the compounds of both steroidal and non-steroidal structures. Suitable non-limiting examples of steroidal anti-inflammatory compounds are corticosteroids such as hydrocortisone, cortisol, hydroxyltriamcinolone, alpha-methyl dexamethasone, dexamethasone-phosphate, beclomethasone dipropionates, clobetasol valerate, desonide, desoxymethasone, desoxycorticosterone acetate, dexamethasone, dichlorisone, diflorasone diacetate, diflucortolone valerate, fluadrenolone, fluclorolone acetonide, fludrocortisone, flumethasone pivalate, fluosinolone acetonide, fluocinonide, flucortine butylesters, fluocortolone, fluprednidene (fluprednylidene) acetate, flurandrenolone, halcinonide, hydrocortisone acetate, hydrocortisone butyrate, methylprednisolone, triamcinolone acetonide, cortisone, cortodoxone, flucetonide, fludrocortisone, difluorosone diacetate, fluradrenolone, fludrocortisone, difluorosone diacetate, fluocinolone, fluradrenolone acetonide, medrysone, amcinafel, amcinafide, betamethasone and the balance of its esters, chloroprednisone, chlorprednisone acetate, clocortelone, clescinolone, dichlorisone, diflurprednate, flucloronide, flunisolide, fluoromethalone, fluperolone, fluprednisolone, hydrocortisone valerate, hydrocortisone cyclopentylpropionate, hydrocortamate, meprednisone, paramethasone, prednisolone, prednisone, beclomethasone dipropionate, triamcinolone. Mixtures of the above steroidal anti-inflammatory compounds can also be used.

Non-limiting example of non-steroidal anti-inflammatory compounds include nabumetone, celecoxib, etodolac, nimesulide, apasone, gold, oxicams, such as piroxicam, isoxicam, meloxicam, tenoxicam, sudoxicam, and CP-14,304; the salicylates, such as aspirin, disalcid, benorylate, trilisate, safapryn, solprin, diflunisal, and fendosal; the acetic acid derivatives, such as diclofenac, fenclofenac, indomethacin, sulindac, tolmetin, isoxepac, furofenac, tiopinac, zidometacin, acematacin, fentiazac, zomepirac, clindanac, oxepinac, felbinac, and ketorolac; the fenamates, such as mefenamic, meclofenamic, flufenamic, niflumic, and tolfenamic acids; the propionic acid derivatives, such as ibuprofen, naproxen, benoxaprofen, flurbiprofen, ketoprofen, fenoprofen, fenbufen, indopropfen, pirprofen, carprofen, oxaprozin, pranoprofen, miroprofen, tioxaprofen, suprofen, alminoprofen, and tiaprofenic; and the pyrazoles, such as phenylbutazone, oxyphenbutazone, feprazone, azapropazone, and trimethazone.

The variety of compounds encompassed by this group are well-known to those skilled in the art. For detailed disclosure of the chemical structure, synthesis, side effects, etc. of non-steroidal anti-inflammatory compounds, reference may be had to standard texts, including Anti-inflammatory and Anti-Rheumatic Drugs, K. D. Rainsford. Vol. I-III, CRC Press, Boca Raton. (1985), and Anti-inflammatory Agents, Chemistry and Pharmacology 1, R. A. Scherrer, et al., Academic Press, New York (1974), each incorporated herein by reference.

Mixtures of these non-steroidal anti-inflammatory compounds may also be employed, as well as the pharmacologically acceptable salts and esters of these compounds.

In addition, so-called “natural” anti-inflammatory compounds are useful in methods of the disclosed invention. Such compounds may suitably be obtained as an extract by suitable physical and/or chemical isolation from natural sources (e.g., plants, fungi, by-products of microorganisms). Suitable non-limiting examples of such compounds include candelilla wax, alpha bisabolol, aloe vera, Manjistha (extracted from plants in the genus Rubia, particularly Rubia Cordifolia), and Guggal (extracted from plants in the genus Commiphora, particularly Commiphora Mukul), kola extract, chamomile, sea whip extract, compounds of the Licorice (the plant genus/species Glycyrrhiza glabra) family, including glycyrrhetic acid, glycyrrhizic acid, and derivatives thereof (e.g., salts and esters). Suitable salts of the foregoing compounds include metal and ammonium salts. Suitable esters include C₂-C₂₄ saturated or unsaturated esters of the acids, preferably C₁₀-C₂₄, more preferably C₁₆-C₂₄. Specific examples of the foregoing include oil soluble licorice extract, the glycyrrhizic and glycyrrhetic acids themselves, monoammonium glycyrrhizinate, monopotassium glycyrrhizinate, dipotassium glycyrrhizinate, 1-beta-glycyrrhetic acid, stearyl glycyrrhetinate, and 3-stearyloxy-glycyrrhetinic acid, and disodium 3-succinyloxy-beta-glycyrrhetinate.

Generally, anti-inflammatory non-steroid drugs are included in the definition of “analgesics” because they provide pain relief. However, in this disclosure, anti-inflammatory non-steroid drugs are included in the definition of anti-inflammatory compounds. Accordingly, the definition of the term “analgesics” for the purposes of the current disclosure does not include anti-inflammatory compounds. Thus, suitable analgesics include other types of compounds, such as, for example, opioids (such as, for example, morphine and naloxone), local anaesthetics (such as, for example, lidocaine), glutamate receptor antagonists, α-adrenoreceptor agonists, adenosine, canabinoids, cholinergic and GABA receptors agonists, and different neuropeptides. A detailed discussion of different analgesics is provided in Sawynok et al., (2003) Pharmacological Reviews, 55:1-20, the content of which is incorporated herein by reference.

Suitable antibiotics include, without limitation nitroimidazole antibiotics, tetracyclines, penicillins, cephalosporins, carbopenems, aminoglycosides, macrolide antibiotics, lincosamide antibiotics, 4-quinolones, rifamycins and nitrofurantoin. Suitable specific compounds include, without limitation, ampicillin, amoxicillin, benzylpenicillin, phenoxymethylpenicillin, bacampicillin, pivampicillin, carbenicillin, cloxacillin, cyclacillin, dicloxacillin, methicillin, oxacillin, piperacillin, ticarcillin, flucloxacillin, cefuroxime, cefetamet, cefetrame, cefixine, cefoxitin, ceftazidime, ceftizoxime, latamoxef, cefoperazone, ceftriaxone, cefsulodin, cefotaxime, cephalexin, cefaclor, cefadroxil, cefalothin, cefazolin, cefpodoxime, ceftibuten, aztreonam, tigemonam, erythromycin, dirithromycin, roxithromycin, azithromycin, clarithromycin, clindamycin, paldimycin, lincomycirl, vancomycin, spectinomycin, tobramycin, paromomycin, metronidazole, tinidazole, ornidazole, amifloxacin, cinoxacin, ciprofloxacin, difloxacin, enoxacin, fleroxacin, norfloxacin, ofloxacin, temafloxacin, doxycycline, minocycline, tetracycline, chlortetracycline, oxytetracycline, methacycline, rolitetracyclin, nitrofurantoin, nalidixic acid, gentamicin, rifampicin, amikacin, netilmicin, imipenem, cilastatin, chloramphenicol, furazolidone, nifuroxazide, sulfadiazin, sulfametoxazol, bismuth subsalicylate, colloidal bismuth subcitrate, gramicidin, mecillinam, cloxiquine, chlorhexidine, dichlorobenzylalcohol, methyl-2-pentylphenol or any combination thereof.

Further, least one reagent which prevents the breakdown of extracellular matrix may be added to the composition or delivered with the composition or a short time (e.g., within 8 hours, or within 4 hours, or within 2 hours or within 1 hour) before or after administration of the composition of the present invention. In one embodiment of the invention, the at least one reagent is a matrix metalloproteinase (MMP) downregulating agent. Examples of suitable MMP down-regulating agents are well known in the art and include, without limitations ONO-4817, Tissue Inhibitor of Metalloproteinase-1 (TIMP-1), Tissue Inhibitor of Metalloproteinase-2 (TIMP-2), Tissue Inhibitor of Metalloproteinase-3 (TIMP-3), Tissue Inhibitor of Metalloproteinase-4 (TIMP-4), Chemically modified tetracycline-3 (CMT-3), 5-amino-2-mercapto-1,3,4-thiadiazole based inhibitors of matrix metalloproteinases, Docetaxel, Quercetin, Green tea extract, TNF-α inhibitors, IL-1β inhibitors, p38 inhibitors, prinomastat, P16, Isoflavones, PCK3145, and any combinations thereof. For a more detailed description of these compounds, as well as suitable non-limiting treatment protocols, see, e.g., Kim et al., Inhibition of Matrix Metalloproteinase-9 Prevents Neutrophilic Inflammation in Ventilator-Induced Lung Injury, AM. J. PHYSIOL. LUNG CELL MOL PHYSIOL, May 12, 2006, Epub ahead of print. Jamloki et al., QSAR analysis of some 5-amino-2-mercapto-1,3,4-thiadiazole based inhibitors of matrix metalloproteinases and bacterial collagenase, BIOORG MED CHEM LETT. 2006 May 6, Epub ahead of print. Li et al., Antitumor and antimetastatic activities of docetaxel are enhanced by genistein through regulation of osteoprotegerin/receptor activator of nuclear factor-kappaB (RANK)/RANK ligand/MMP-9 signaling in prostate cancer, CANCER RES. 66(9):4816-25 (2006); Vijayababu et al., Quercetin downregulates matrix metalloproteinases 2 and 9 proteins expression in prostate cancer cells (PC-3), MOL CELL BIOCHEM. 2006 Apr. 28, Epub ahead of print. Roomi et al., In vivo and in vitro antitumor effect of ascorbic acid, lysine, proline, arginine, and green tea extract on human fibrosarcoma cells HT-1080, MED ONCOL. 23:105-11 (2006); Sang et al., Matrix metalloproteinase inhibitors as prospective agents for the prevention and treatment of cardiovascular and neoplastic diseases, CURR TOP MED CHEM. 6:289-316 (2006); Puli et al., Inhibition of matrix degrading enzymes and invasion in human glioblastoma (U87MG) Cells by isoflavones, J NEUROONCOL. 2006 Apr. 6, Epub ahead of print, Wang et al., P16 inhibits matrix metalloproteinase-2 expression via suppression of Sp1-mediated gene transcription, J CELL PHYSIOL. 208:246-52 (2006); Annabi et al., Inhibition of MMP-9 secretion by the anti-metastatic PSP94-derived peptide PCK3145 requires cell surface laminin receptor signaling, ANTICANCER DRUGS, 17:429-438 (2006).

In another aspect, the invention provides a method of treatment a chondrocyte-derived tissue comprising administering to a subject in need thereof an effective amount of the composition according to any of the embodiments described above. Further, the composition may be formulated with a pharmaceutically acceptable carrier or diluent. In one embodiment, the pharmaceutically acceptable carrier or diluent is liquid or semi-solid. Further, the formulation may be suitable for intramuscular, intravenous, intramedullary, or intraarticular injection.

The composition may be delivered by several means, including, without limitation, an injection into the desired part of the subject's body (e.g., joint or intervertebral disk), surgical placement, intramuscular, intravenous, intramedullary, or intraarticular injection, or any combination thereof. The surgical placement is especially suitable for the cells grown on a scaffold and thus formed into a suitable shape.

In yet another embodiment, the invention provides a method of treatment a chondrocyte-derived tissue, comprising administering to a subject in need thereof an effective amount of a nucleic acid sequence comprising a nucleic acid sequence encoding the at least one bioactive factor. In this embodiment, the nucleic acid sequence comprising the nucleic acid sequence encoding the at least one bioactive factor may be delivered by a direct injection into the part of the subject body which is in need of the treatment. Alternatively, the nucleic acid sequence may be distributed from a depot located at the part of the subject body which is in need of the treatment. Further, as described above, the nucleic acid sequence comprising the nucleic acid sequence encoding at least one bioactive factor.

### EXAMPLES

### Example 1

Without wishing to be bound by a single theory, if transplanted bone marrow cells can be induced by LMP-1 to produce other BMPs, such as BMP-2, BMP-7 and BMP-9, or even disc matrix, then these cells may represent a practical source of cells for ex vivo therapy of disc degeneration. As discussed below, Applicants carried out experiments to test whether marrow cells from a human donor can be stimulated to produce proteoglycans and BMPs by overexpressing LMP-1.

### Preparation of Human Bone Marrow Cells

Human bone marrow and matched peripheral blood were collected from 3 females (aged 21, 25 and 35 years of age) through Cambrex Bio Science Walkersville INC. The studies were approved by the human subjects Institutional Review Board. The bone marrow was obtained from the posterior iliac crest with a marrow biopsy needle. Multiple passes were performed to aspirate a 30 cm³ volume of marrow fluid in Heparin as an anti-coagulant. In addition, approximately 60 cm³ of peripheral blood was obtained from the same donor using citrate as an anti-coagulant. Within 24 hours of harvest, the marrow and peripheral blood were mixed and centrifuged in a Magellan system (Medtronic Sofamor Danek, Memphis, Tenn.). The cells were spun in accordance with the standard instrument protocol. The concentrated human bone marrow cells were withdrawn, which included the mesenchymal cell enriched layer. Cell numbers were determined by counting a control well using a hemocytometer and Hemavit count (CDC Tech INC, Oxford, Conn.).

Cell culture and Ad-f35-LMP1 Adenovirus Transfection

A replication deficient type 5 adenovirus with serotype 35 fiber (F 35) carrying the human LMP-1 cDNA driven by the CMV promoter was provided by Medtronic Sofamor Inc. This chimeric adenovirus is capable of infecting human cells through a mechanism independent of the CAR receptor and is thought to have higher infectivity. The viral dose was expressed as a multiplicity of infection (MOI), the number of plaque forming units (pfu) per cell.

Concentrated marrow cells were infected with different MOIs (3.3, 10, 33, and 100) of the Ad-f35-LMP1 virus at 37° C. for a period of 30 minutes in a 0.5 ml of serum free AMEM/F 12 mesenchymal stem cell medium (Cambrex, Walkersville, Md.). A no virus group served as a control. 400,000 cells in each tube and six tubes for each treatment group and control group were prepared. The cells were then plated onto 6 well plates with additional 1.5 mls of media containing 10% fetal bovine serum (FBS) cells at 400,000 cells per well and 6 wells for each treatment group and control group. The cells were incubated at 37° C. in 5% CO2 with humidification for 6 days. At day six, the cells and media of three wells in each treatment groups and control group were combined. After centrifugation, the media were collect for proteoglycan assay and cells for RNA extraction.

### Proteoglycan Assay

The proteoglycan (PG) content of the culture media was assayed using the 1, 9 dimethylmethylene blue (DMMB) assay. Twenty micro-liters (20 μl) of medium from each well were mixed gently with 200 μl DMMB dye solution in a 96-well micro-titer plate, and the optical density (OD) was checked immediately at 520 nm wavelength. A standard curve was constructed using serial dilutions of chondroitin sulfate (Sigma Chemical, St. Louis, Mo.). PG content in the media was defined as fold increase in Ad0 5-LMP1 treated samples over control.

Reverse Transcription and Real time PCR

RNA was isolated at day 6 using RNAqueous Kit as specified by the manufacturer (Ambion inc., Austin, Tex., U.S.A.). The RNA was treated with DNAse (Ambion, Inc., Austin, Tex., U.S.A.) to remove DNA contamination of the samples. The concentration of the isolated RNA was determined with RNA 6000 Nano Assay Protocol (Agilent Technologies, Waldbronn, Germany). Reverse transcription was carried out in 100 up volume with 500 ng of total RNA using Reverse Transcription reagents (Applied Biosystem, Foster city, CA): 2.5 up of 50 U/μl Multiscribe reverse transcriptase; 2 μl of 20 U/μl Rnase inhibitor; 22 μl of 25 mM MgCl2 solution; 5 up of 50 uM Random Hexamers; 10 μl of IOX PCR Buffer II and 20 gl of 12.5 mM dNTP mix with dUTP. The reaction conditions were 10 minutes at 25 C, 30 minutes at 48 C and 5 minutes at 95 C. To confirm the absence of DNA contamination, RNA samples treated without reverse transcriptase were also subjected to PCR: the absence of PCR product confirmed the lack of DNA contamination.

mRNA levels of LMP-1, aggrecan, BMP-2, BMP-7 and BMP-9 were determined with a real-time PCR method using SYBR Green Real-Time PCR Kit (Applied Biosystem, Foster City, Calif.). Twenty-five microliters (25 μl) of reaction volume included 5 μl of cDNA of each sample, 3.75 picomole of primer, and 12.5 up of SYBR Green master mix. Real-time PCR was performed with the following 3-step protocol; step 1: 50° C. for 2 minutes, step 2: 95 C for 10 minutes, and step 3: (95 C for 15 seconds, 60 C for 1 minute)×40 cycles using the Gene Amp; 5700 Sequence Detection system (Applied Biosystem, Foster City, Calif.). To confirm amplification specificity, the PCR products were subjected to a dissociation curve analysis. Threshold cycles (Ct) of each reaction was standardized according to 18S using the comparative ^{-ΔΔ}Ct method, as described previously. See S. T. Yoon et al., ISSLS Prize Winner: LMP-1 Upregulates Intervertebral Disc Cell Production of Proteoglycans and BMPs In Vitro and In Vivo, 29 SPINE 2603-11 (2004). The primers for all of the genes were validated by determining the product size on an agarose gel and by DNA sequencing of the amplicon.

### Statistical Analysis

The entire experiment was repeated six times with three different human donors with similar results. All data is presented as a ratio over control. Two-tailed student tests were used to calculate p values. The data are presented as mean ±SEM. P<0.05 was used as cut off point for statistical significance.

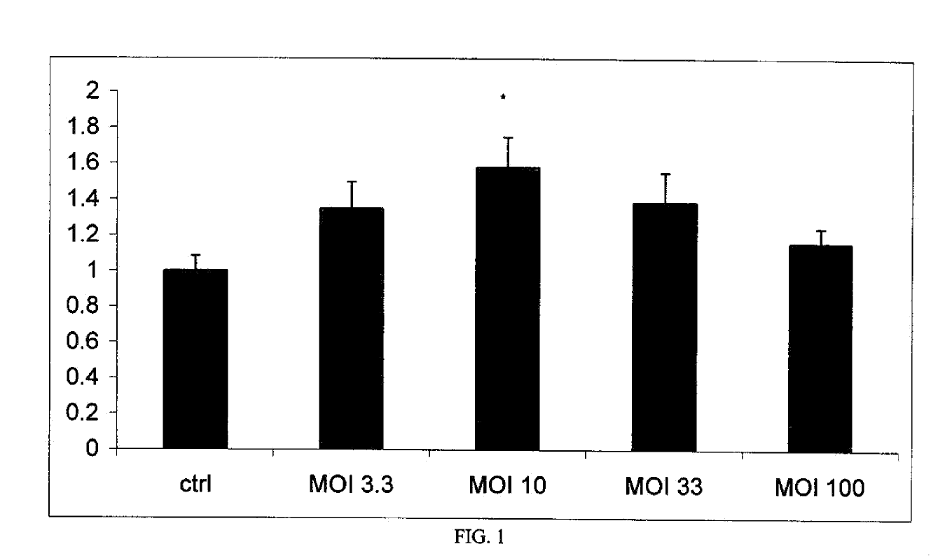

DMMB assay showed that compared to the control group, the proteoglycan production was increased to 1.35, 1.58, 1.39 and 1.46 fold at MOI 3.3, 10, 33 and 100 respectively (FIG. 1). There was a significant increase at MOI 10 (P<0.05).

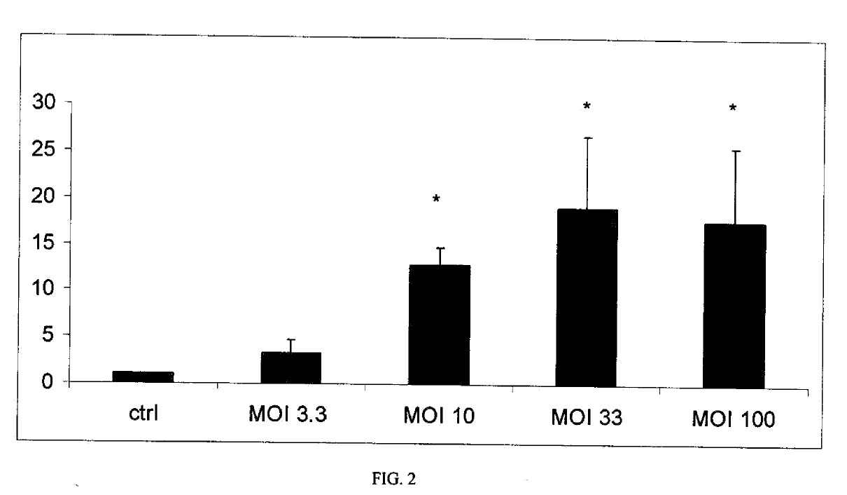

Real time PCR data showed that, compared to the control group, the mRNA levels of LMP-1 were increased in a dose-dependent manner; 3.28, 12.79, 18.99 and 17.65 fold at MOI 3.3, 10, 33 and 100 respectively; the increases were significant at MOI 10, 33 and 100 (P<0.05) (FIG. 2).

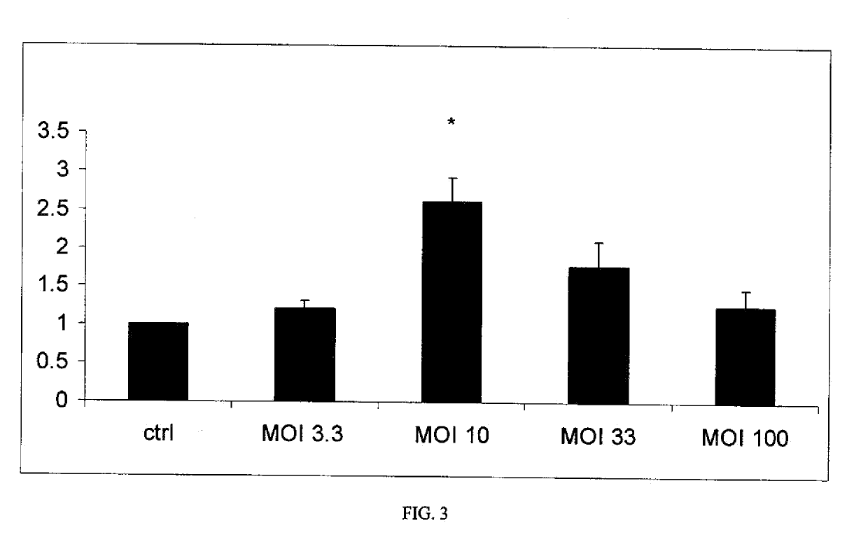

Compared to the control group, the mRNA levels of aggrecan increased 1.21, 2.61, 1.78 and 1.26 fold at MOI 3.3, 10, 33 and 100 respectively; the increase was significant at MOI 10 (P<0.05) (FIG. 3).

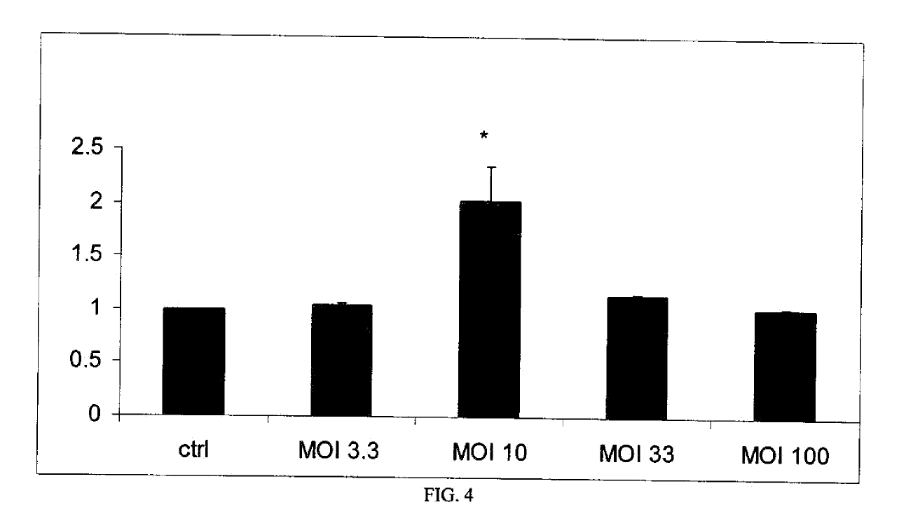

Compared to the control group, the mRNA levels of BMP-2 were increased 1.05, 2.03, 1.15 and 1.02 fold at MOI 3.3, 10, 33 and 100 respectively; the increase was significant at MOI 10 (P<0.05) (FIG. 4).

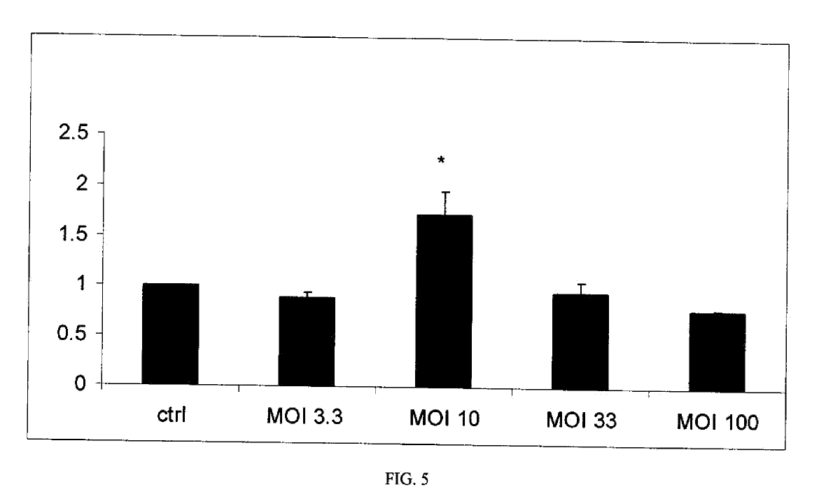

Compared to the control group, the mRNA levels of BMP-7 were 0.88, 1.72, 0.95 and 0.78 fold at MOI 3.3, 10, 33 and 100 respectively; the increase was significant at MOI 10 (P<0.05) (FIG. 5).

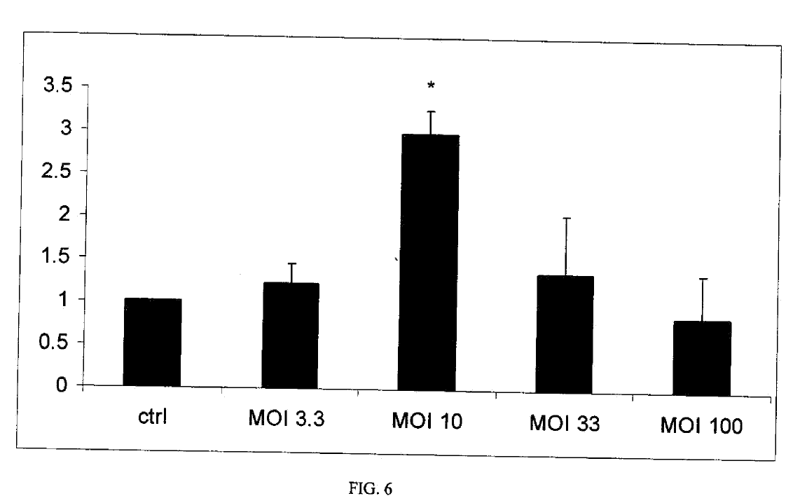

Finally, compared to the control group, the mRNA levels of BMP-9 were 1.23, 2.98, 1.36 and 0.87 fold at MOI 3.3, 10, 33 and 100 respectively; the increase was significant at MOI 10 (P<0.05) (FIG. 6).

### CONCLUSIONS

It has already been shown that overexpression of LMP-1 increases proteoglycan production by upregulating BMP-2 and BMP-7 in rabbit disc cells. The study presented here shows a very similar result in non-disc cells, in particular, using human bone marrow cells.

In addition, BMP-9 mRNA levels upregulated by LMP-1 was first detected in this study. It has been reported that BMP-2 and BMP-9 promoted chondrogenic differentiation of human multipotential mesenchymal cells and overcame the inhibitory effect of IL-1. See M. K. Majumdar et al., BMP-2 and BMP-9 Promotes Chondrogenic Differentiation of Human Multipotential Mesenchymal Cells and Overcomes the Inhibitory Effect of IL-1, 189 J. CELL. PHYSIOL. 275-84 (2001). Therefore, the upregulation of multiple BMPs plays an important role in the synthesis of proteoglycan and leads to the chondrogenic differentiation of human marrow cells.

In summary, human bone marrow cells have the potential to be induced by LMP-1 to increase proteoglycan synthesis by upregulating multiple BMPs. Thus, these cells are good candidates for cell therapy and gene therapy in disc degeneration, including but not limited to ex vivo gene therapy.

The patent and scientific literature referred to herein establishes the knowledge that is available to those with skill in the art. All United States patents and published or unpublished United States patent applications cited herein, except for those cited in the first paragraph of this application, are incorporated by reference. All published foreign patents and patent applications cited herein, except for those cited in the first paragraph of this application, are hereby incorporated by reference. All other published references, documents, manuscripts and scientific literature cited herein are hereby incorporated by reference.

While this invention has been particularly shown and described with references to preferred embodiments thereof, it will be understood by those skilled in the art that various changes in form and details may be made therein without departing from the scope of the invention encompassed by the claims.

What is claimed is:

1. A composition comprising a substantially purified plurality of cells artificially enhanced with at least one bioactive factor capable of causing at least a portion of the plurality of cells to express an altered amount of at least one chondrogenic marker, wherein the at least one bioactive factor comprises a fragment of a LIM mineralization protein (LMP) necessary for osteoinduction or encodes an amino acid sequence comprising the fragment of an LMP protein necessary for osteoinduction.

2. The composition of claim 1, wherein the substantially purified plurality of cells comprises at least one multipotent cell.

3. The composition of claim 2, wherein at least one pluripotent cell is a mesenchymal cell.

4. The composition of claim 1 wherein members of the substantially purified plurality of cells are human cells.

5. The composition of claim 4, wherein the human cells are harvested from an allograft source.

6. The composition of claim 1, wherein the at least one bioactive factor is LMP-1, LMP-3, or LMP-1s.

7. The composition of claim 1, wherein the at least one bioactive factor is LMP-1.

8. The composition of claim 1 further comprising at least one additive selected from the group consisting of lubricants, anti-inflammatory agents, antibiotics, analgesics and any combinations thereof.

9. A composition comprising a substantially purified plurality of cells enhanced with at least one bioactive factor capable of causing at least a portion of the plurality of cells to differentiate into chondrogenic or chondrogenic-like cells, wherein the at least one bioactive factor comprises a fragment of a LIM mineralization protein (LMP) necessary for osteoinduction or encodes an amino acid sequence comprising the fragment of an LMP protein necessary for osteoinduction.

10. The composition of claim 9, wherein the substantially purified plurality of cells comprises at least one multipotent cell.

11. The composition of claim 10, wherein at least one pluripotent cell is a mesenchymal cell.

12. The composition of claim 9 wherein members of the substantially purified plurality of cells are human cells.

13. The composition of claim 12, wherein the human cells are harvested from an allogeneic source.

14. The composition of claim 9, wherein the at least one bioactive factor is LMP-1, LMP-3 or LMP-1s.

15. The composition of claim 14, wherein the at least one bioactive factor is LMP-1.

16. The composition of claim 9 further comprising at least one additive selected from the group consisting of lubricants, anti-inflammatory agents, antibiotics, analgesics and any combinations thereof.

17. A formulation comprising the composition of claim 1 with a pharmaceutically acceptable carrier or diluent.

18. The formulation of claim 17, wherein the pharmaceutically acceptable carrier or diluent is liquid or semi-solid.

19. The formulation of claim 17 suitable for intramuscular, intravenous, intramedullary, or intraarticular injection.
